(19) United States
(12) Patent Application Publication
(10) Pub. No.: US 2026/0141990 A1
(43) Pub. Date: May 21, 2026

(54) SCALING SPECTRAL SEARCH FOR MASS SPECTROMETRY DATA

(71) Applicant: DH Technologies Development Pte. Ltd., Singapore (SG)

(72) Inventors: Stephen TATE, Barrie (CA); Cristiano VEIGA, Toronto (CA)

(73) Assignee: DH Technologies Development Pte. Ltd., Singapore (SG)

(21) Appl. No.: 19/123,149

(22) PCT Filed: Oct. 19, 2023
(86) PCT No.: PCT/IB2023/060580

§ 371 Date: Apr. 22, 2025

Related U.S. Application Data
(60) Provisional application No. 63/418,766, filed on Oct. 24, 2022.

(51) Int. Cl. G16C 20/90; G16C 20/20

(52) U.S. Cl. CPC G16C 20/90; G16C 20/20

(57) ABSTRACT

A method and system for investigating and analyzing data from a mass spectrometer, the system including a spectral database, a count service for receiving scan data generated by the mass spectrometer and identifying a number of spectra in the data, a scaling service for receiving the scan data generated by the mass spectrometer, receiving the number of spectra from the count service, and initiating a plurality of query services, each query service of the plurality of query services corresponding to at least one spectra of the number of spectra and for querying the spectral database with the corresponding at least one spectra and returning a match between the corresponding at least one spectra and at least one known spectra from the spectral database, and a results service for retrieving each match, and formatting each match into an output data structure.

### CROSS-REFERENCE TO RELATED APPLICATIONS

This application is being filed on Oct. 19, 2023, as a PCT International Patent Application that claims priority to and the benefit of U.S. Provisional Application No. 63/418,766, filed on Oct. 24, 2022, which is hereby incorporated by reference in its entirety.

### INTRODUCTION

Mass spectrometry (MS) is method of compound analysis that provides a measurement of the mass-to-charge ratio of ions in the compound. The results are presented as a mass spectrum, which plots the intensity of the particular ions as a function of the mass-to-charge ratio. A Spectral Library search provides a method for the identification of the compounds in the mass spectrum according to the pattern of the ions on the mass spectrum. The size and expanse of the Spectral Libraries is now exceeding what is storable on desktop systems and the level of spectra which require searching is also increasing. In the case of Metabolomic workflows there are no tandem mass spectrometry (MS/MS or MS2) based search engines other than traditional spectral library searching. Due to this, analysis of MS2 data can be prohibitively time-and resource-consuming for analysis systems.

### SUMMARY

In a first aspect, the technology of the present disclosure relates to an analysis system for investigating data from a mass spectrometer, the system including a spectral database, a count service for receiving scan data generated by the mass spectrometer, and identifying a number of spectra in the data, a scaling service for receiving the scan data generated by the mass spectrometer, receiving the number of spectra from the count service, and initiating a plurality of query services, each query service of the plurality of query services corresponding to at least one spectra of the number of spectra, each query service of the plurality of query services for querying the spectral database with the corresponding at least one spectra, and returning a match between the corresponding at least one spectra and at least one known spectra from the spectral database, and a results service for retrieving each match, and formatting each match into an output data structure.

In an example of the above aspect, the system further includes a scan output database for receiving the scan data generated by the mass spectrometer. In another example, the scan data generated by the mass spectrometer is a plurality of key-value pairs. In a further example, each of the plurality of key-value pairs includes a scan and an offset.

In other examples of the above aspect, the spectral database is a document-based database. In another example, the count service performs signal processing on the scan data generated by the mass spectrometer. For example, the count service executes a peak extraction on the data collected by the mass spectrometer such that the number of spectra coincides with a number of peaks in the data. In a further example, the at least one spectra of the number of spectra is only one spectra of the number of spectra. In another example, the match comprises a fit value. In yet another example, the match comprises a purity value.

In still other examples of the above aspect, the system further includes a results database, wherein each query service stores the match corresponding to its at least one spectra in the results database. For example, the results service stores the output data structure in the results database. For a further example, the system further includes a scan output database for receiving the scan data generated by the mass spectrometer, the scan data comprises a plurality of paired sets of a scan and an offset, and the results database communicates with the scan output database to associate each match with one paired set of the plurality of paired sets of a scan and an offset. As another example, each query service, in response to storing the match associated with its at least one spectra in the results database, is reclaimed by the scaling service.

In other examples of the above aspect, formatting each match into an output data structure includes extract-transform-load processing. In another example, the mass spectrometer is a tandem mass spectrometry system.

In another aspect, the technology of the present disclosure relates to a method of analysis of data collected by a mass spectrometer, the method including receiving data collected by the mass spectrometer, counting a number of spectra in the data, initiating a plurality of query services, each query service of the plurality of query services corresponding to at least one spectra of the number of spectra, querying, by each of the plurality of query services, a spectral database, returning, by each of the plurality of query services, at least one match between the corresponding at least one spectra and a known spectra from the spectral database, formatting each match from each of the plurality of query services into an output data structure, and storing the output data structure in a results database.

In an example of the above aspect, the mass spectrometer is a tandem mass spectrometry system. In another example, the method further includes storing, by each of the query services, the match associated with its at least one spectra in the results database, and reclaiming, by the scaling service, each of the plurality of query services. In a further example, each match from each of the plurality of query services comprises at least one of a fit value and a purity value.

The details of one or more techniques are set forth in the accompanying drawings and the description below. Other features, objects, and advantages of these techniques will be apparent from the description, drawings, and claims.

### BRIEF DESCRIPTION OF THE DRAWINGS

FIG. 1 is a block diagram of an example system, according to various examples of the present disclosure.

FIG. 2A is an example dataflow and procedure diagram for analysis of mass spectrometry data, according to various examples of the present disclosure.

FIG. 2B is a flowchart of an example process that is executed by the system of FIG. 1.

FIG. 3A is a diagram of an example network or cloud system in which various examples of the present disclosure may be implemented.

FIG. 3B is a block diagram of an example cloud computing node in which various examples of the present disclosure may be implemented.

Before one or more examples of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DETAILED DESCRIPTION

The present disclosure relates the field of mass spectrometry and more particularly relates to the field of mass spectrometry software, and more particularly relates to the field of mass spectrometry data analytic software.

The present disclosure is directed to methods, systems and computer program products for mass spectrometry, in particular, sample analysis, analyte identification, mass spectrometry data processing, and sample identity prediction, which are now described herein in terms of an example microservice analysis system that provides for high-speed processing of complex mass spectrometry scan output data. The present disclosure is directed to systems and methods for analyzing a readout or data output by a mass spectrometer or mass spectrometry system following a sample analysis. In some aspects, the present disclosure describes systems and methods for high-speed analysis of the data output by a mass spectrometer.

In further aspects, the present disclosure describes systems and methods for distributed searching of a spectral library or database. In still other aspects, the present disclosure relates to a scaling analytical system and distributed querying of a large database. This description is not intended to limit the application of the disclosed technology to the examples presented herein. In fact, after reading the following description, it will be apparent to one skilled in the relevant art(s) how to implement the following examples in alternative implementations (e.g., where the system is implemented in a desktop or other local device or system, where the system is distributed between local and remote networks, etc.). In addition, it will be apparent to one skilled in the relevant art how to implement the following invention in alternative contexts, involving, for example, data other than mass spectrometry scans, such as other complex biological analyses producing large and diverse datasets.

In addition, not all of the components described herein are required to practice the invention, and variations in the arrangement and type of the components may be made without departing from the spirit or scope of the invention. As used herein, the terms “component” and “service” are applied to describe a specific structure for performing specific associated functions, such as a special purpose computer as programmed to perform algorithms (e.g., processes) disclosed herein. The component (or service) can take any of a variety of structural forms, including: instructions executable to perform algorithms to achieve a desired result, one or more processors (e.g., virtual or physical processors) executing instructions to perform algorithms to achieve a desired result, or one or more devices operating to perform algorithms to achieve a desired result.

Various examples will be described in detail with reference to the drawings, wherein like reference numerals represent like parts and assemblies throughout the several views. Reference to various examples does not limit the scope of the claims attached hereto. Additionally, any examples set forth in this specification are not intended to be limiting and merely set forth some of the many possible aspects for the appended claims.

Mass spectrometry (MS) is widely used to determine the molecular mass and elucidate the chemical structures of analytes in a sample. However, depending on the experimental methodology and the sample analyzed, output datasets from mass spectrometry data can contain up to tens of thousands of ions/peaks and features thereof. In general, it is very unlikely that a mass spectrum of a sample would have only one single ion per one analyte. For example, a pure standard analyte Nicotinamide adenine dinucleotide [NAD] analyzed by liquid chromatography-mass spectrometry (LC-MS) can derive various ion species and ion products therefrom. These ion species or ion products derived from NAD could be identified in the mass spectrum of NAD, including the [M+H]+, [M+Na]+, [M+H+H]2+, other adducts, dimers, oligomers, and internal fragments with one or multiple charge states.

Spectral library searching provides a method for the compound identification. The size and expanse of the spectral libraries is now exceeding what can be stored on desktop systems and the number and level of spectra which require searching and identification is also increasing. For example, in the case of metabolomic workflows there are no MS2 (MS/MS or tandem mass spectrometry) based search engines other than spectral library searching. Based on the type of query and performance needs of the spectral library search, various cloud-and cluster-based solutions have been implemented widely in recent years to handle and distribute the heavy computing loads create by large data files, but are often restricted in the complexity of the tasks they can effectively execute.

Mass spectrometry produces data files that are not only large but require complex analysis to produce a useful output. The time and resources required by known systems to perform this analysis are prohibitive. For example, common cloud-or cluster-based approaches, such as hosting the library across a distributed database, quickly become too elaborate for effective use in the context of mass spectrometry data due to the size and complexity of the data. This results in reduced performance of the overall system and large time lags when awaiting results. Though cloud computing has a few established engines for the development of complex workflows, none provide the appropriate speed and accuracy for an effective analysis of complex mass spectrometry data.

Current considerations comparing desktop versus cloud-based processing speeds have mainly focused on small library sizes (e.g., <1 GB) and simplistic cloud scaling. As open-source libraries are increasing in size every year, the need for a system for rapid library access with inherent scalability becomes more and more important. Existing methods have unsatisfactory performance limits or waste resources unnecessarily. The disclosed solution effectively optimizes the system for the large-scale and complex analysis required by mass spectrometry data.

Disclosed herein are systems and method to address these and other limitations in mass spectrometry analysis. This disclosed system and methods provide for distribution of services required for the analysis of data, such as through a microservice architecture, allowing for simultaneous and near real-time analysis of all or numerous sub-spectra of a complex mass spectrometry readout. Though the examples and context discussed herein revolve around mass spectrometry, those of skill in the art will recognize that the concepts disclosed may be applied in other contexts for effective, high-speed, and complex analysis of large data. The disclosed system and method enable decreased processing time as well as the ability to search large libraries or multiple libraries simultaneously in a reduced time frame. The disclosed solution also supports multiple users and has the ability to adjust the compute resources consumed to achieve any required performance at the lowest cost. The disclosed solution allows for better support of larger spectral libraries, which is currently a major struggle for existing library searching solutions. It also presents the potential for enormous performance gains by leveraging the massive scalability of the cloud and microservices to generate results in near real time.

In a microservice architecture, a software application is structured as a collection of small, well-defined microservices. Each microservice corresponds to an action performed by an application. The microservices communicate with each other only through well-defined application programming interfaces (APIs). Each functionality of the software application is its own dedicated microservice. A monolithic architecture has drawbacks particularly when the software application grows and the number of developers working on the application grows. Splitting up the application into more maintainable modules (or microservices) can improve efficiency by allowing each module to be worked on independently.

In a microservices architecture type software application, the application consists of a computing pipeline or workflow having a number of microservice functionalities or actions that are performed in sequences or loops. In examples, the microservices can be grouped into different services. In examples, each service uses a different cluster of computing resources hosted by the cloud computing platform.

Disclosed herein are mass spectrometry-based workflows which enable near real time computation of results even from the most complex of mass spectrometry data, e.g., metabolomics. The solution to the noted problems with mass spectrometry analysis and cloud-computing limitations is to implement a light weight microservice based architecture which allows for the rapid scaling of processing units. This method could be a workflow, which may, for example, be defined using common workflow language (CWL) or Argo workflows for the definition of the workflow. The disclosed method and system enable the monitoring of a queue to determine if processing requires scaling and the degree to which scaling should be executed for optimal speed and accuracy in the analysis of the spectra. The method and system described herein provide for determining optimal resource use within the processing unit and efficient scaling if a unit is overused. Disclosed herein is a method for the definition of the required scalable units. The system disclosed herein may continually monitor the workflow to determine the optimal scalable definition is in place. The method and system may also provide a mechanism for the removal and scale in of resources as needed.

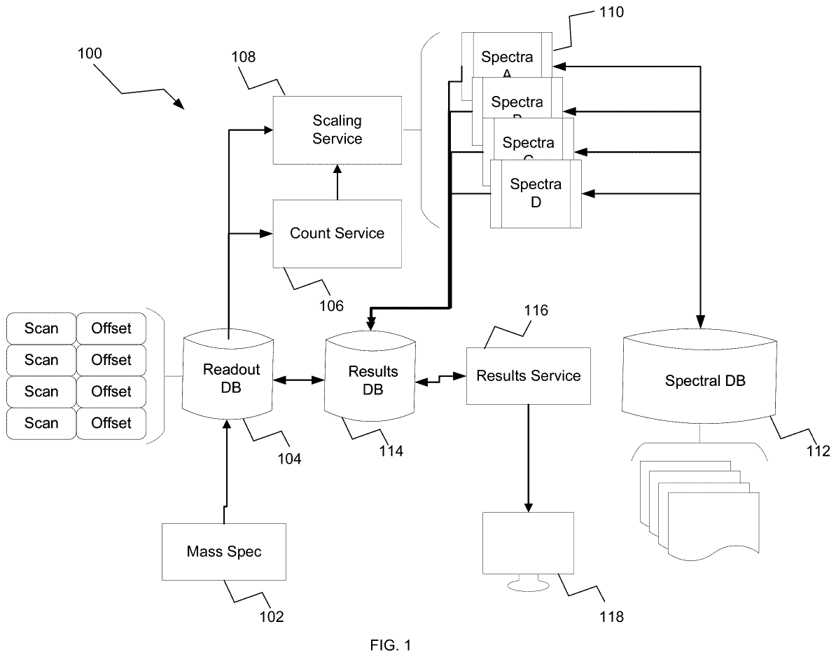

Referring now to FIG. 1, a block diagram of an example system 100 for spectral analysis of mass spectrometry data is shown. System 100 may generally be used for predicting a sample identity, identifying analytes of a sample, dividing a readout into analyte components, predicting or identifying the analyte components, or any combination thereof. Example system 100 includes one or more mass spectrometers 102, a readout database 104, a count service 106, a scaling service 108, a plurality of query services 110, a spectral database 112, a results database 114, a results service 116, and a user interface 118.

System 100 may be a microservice system, with the analysis performed by a collection of loosely coupled services. In examples, the spectrometer 102 or the user interface 118 may be outside of the system 100 and communicate with the system 100 via a gateway, such as a gateway API, or another reverse proxy. System 100 may be a fully cloud-based system or instead by based on a locally sourced server or distributed across local, remote, and cloud-based servers.

In some aspects of the present disclosure, implementing system 100 using a microservice architecture may provide advantages through the speed and ease of scalability of such an architecture. Many existing spectral library searches are burdened by a massive backbone infrastructure necessary to support the system distribution. Using a microservice system, which is scaled to an appropriate size based on the size and complexity of the data to be analyzed, system 100 instead scales itself as necessary to accommodate a given size of data to be analyzed. By using a lightweight microservice system that scales up in response to an active analytic load, there is no large baseline infrastructure to constantly maintain with its associated system costs.

Examples of system 100 may further comprise a container orchestration system (not shown). The container orchestration system may be any known container orchestration system and may, in examples, be selected according to a preferred workflow. For example, in examples where an Argo workflow is used, a Kubernetes orchestration system may be used.

Containers are used in cloud computing as an abstraction at the application layer that is run by a single operating system kernel. In a cloud computing environment, multiple containers can run on the same machine, sharing resources with other containers. To handle an increasing workload, new replicas of a smallest deployable computer processing unit (SDCPU) can be deployed. For a decreasing workload, SDCPUs can be shut down as needed. SDCPU refers to the smallest deployable unit of computing that represents a processing power running on a cluster. In examples, a SDCPU is a group of one or more containers, tightly coupled with shared storage and network, that can be replicated.

One or more spectrometers 102 may be a mass spectrometer, a mass spectrometry system, or two or more mass spectrometers in sequence or tandem. Mass spectrometer 102 may also incorporate other relevant devices and analysis. For example, mass spectrometry 102 may be couple to a chromatographic component, such a gas-or liquid chromatography. Mass spectrometer 102 receives a sample and performs mass analysis, such as by measuring a mass-to-charge ratio of one or more molecules present in the sample. In examples performing an MS2 or tandem mass spectrometry analysis, mass spectrometer 102 may output a set of scan data by the first mass spectrometer 102, by the second mass spectrometer 102, or both. In examples, MS2 may involve an additional step, e.g., fragmentation, between the first and second mass spectrometers, and system 100 may be implemented before or after fragmentation.

Mass spectrometer 102 may be an integrated part of system 100 or may be external to the system 100 and instead provide scan output data to the system through another application, service, API, etc. In examples, mass spectrometer 102 may include an interface or application to, for example, serve as a controller the mass spectrometer or for receiving the data output from the mass spectrometer. In such examples, the interface or application may form part of the system 100, while additional components of the mass spectrometer (e.g., ion source, mass analyzer, detector, etc.) are outside of the system 100.

Readout database 104 receives a readout, or a data output, from the one or more spectrometers 102. Readout database 104 provides a working source of the mass spectrometry data for the services of system 100 as the system executes analysis of the data. In examples wherein the acquisition of mass spectrometry data is fully local, readout database 104 may serve as an initiation point for the system 100. Readout database 104 may be in direct communication with mass spectrometer 102 and receive the readout or data output directly from the mass spectrometer. In some cases, such as examples where the mass spectrometer lies fully outside of system 100, readout database 104 may receive the output data through an intermediary traffic manager interface, or another gateway or edge service.

In examples, readout database 104 receives data from the one or more spectrometers 102 in a key-valued pair. Readout database 104 may receive data from the one or more spectrometers 102 in a paired scan and offset. Readout database 104 receives and holds the readout data from the mass spectrometer for ready accessibility by the count service 106 and the scaling service 108. Due the large size of data output from the mass spectrometer, readout database 104 provides for maintenance of the scan data from a particular run of the mass spectrometer and ready access to the scan data for the count service 106 and the scaling service 108 as needed.

In examples, system 100 may further include an initiation service (not shown). The initiation service may serve to receive and initiate workflow requests. The initiation service may provide input from readout database 104 into the initiated workflow. The initiation service may provide workflow management for system 100. In examples, workflow management may be accomplished using Argo workflow automation. It is also envisioned that other container-native or continuous-integration/continuous-deployment engines may be used. The initiation service may be an independent service or container as it generally will not require scaling. In examples, initiation module or its functions may be integrated with another service or container of the system, such as count service 106 or scaling service 108.

Count service 106 receives the readout data from the mass spectrometer 102 via the readout database 104. Count service 106 determines a number of spectra and/or a number of peaks in the scan output. Count service 106 may generally perform a known or contemplated signal processing algorithm to the readout data from the mass spectrometer 102 to determine the number of peaks of spectra in the readout data. Count service 106 may execute any number of known analysis algorithms on the scan output to determine a number of spectra for individual or group identification, such as a peak extraction, a peak finder, or a peak grouping algorithm on the scan output. In examples, count service 106 may be integrated with another service or container in the system 100, such as an initiation or workflow service (not shown) or scaling service 108.

The number of spectra and/or peaks determined by count service 106 is used by scaling service 108 to determine an optimum number of query services 110 to deploy. In examples, another service, such as the count service 106, or an optimization service or an orchestration service, may determine an optimum number of query services 110 to deploy.

Scaling service 108 may receive or retrieve both the scan output data from the readout database 104 and the number of spectra or peaks from the counter service 106. Scaling service 108 may perform scaling by entering a map state or another state or process for running a set of steps of each element of an input array.

Scaling service 108 may initiate an individual query service 110 for each single spectra identified by the count service 106. In examples, count service 106 may also perform grouping functions, such as a peak grouping function, and scaling service 108 may instead initiate an individual query service 110 for each group of spectra identified by the count service 106. In examples, count service 106 may not be a separate service and may instead be integrated with the scaling service 108.

Query services 110 generally comprise a plurality of services spun out by the scaling service 108 based on the number of spectra identified by the count service. Each query service 110 receives, from the scaling service, at least one spectra of the number of spectra identified by the count service. In examples, a particular query service 110 may receive only one spectra or one peak. In examples, a particular query service 110 may receive a spectra with two or more peaks. In examples, a particular query service 110 may receive two or more spectra. Each query service 110 may include one or more SDCPU, as required for the size of the spectra received by a particular query service 110.

Each query service 110 submits a query to the spectral database 112 for its particular spectra or peaks. Each query service 110 then receives or retrieves from spectral database 112 a match for the spectra submitted. The match may be a simple comparison and match between a scan-offset pair of the submitted spectra and a scan-offset pair associated with a particular known spectra file in the spectral database 112. The match may be a comparison and match be a scan-offset pair of the submitted spectra and a scan-offset pair associated with a different spectra. The different spectra may be a known spectra file or may be a spectra associated with a different scan or query or a previous scan or query. The different spectra may be an unknown spectra from a previous or concurrent scan or query. The match may be between a scan-offset pair of the submitted spectra and an unknown scan-offset pair. Query services 110 may evaluate the returned match, such as by assessing whether the returned match meets a threshold level of similarity to the submitted spectra. In examples, the match may be a fit value or a purity value, or both. Fit and purity are both common measures for spectral matches and many algorithms for their determination are known in the art. Matches may also constitute any number of other algorithms or means for determining and evaluating potential matches for a given spectra including, but not limited to, cos angle analysis and machine learning systems to identify spectral matches.

Because each query service 110 is able to simultaneously query the spectral database 112, the system is able to operate on all the data from the scan output at once. Each query service 110, when the match is received or retrieved from the spectral database 112, sends the match to the results database 114 to be read into a standard output data structure by the results service 116. Once a particular query service 110 stores its retrieved match in the results database 114, that query service 110 may reintegrate with or be reclaimed by scaling service 108.

Spectral database 112 comprises a collection of known spectra and their associated data. Database read issues are a frequent cause of slowing down system processing speeds and increasing the total time to produce a final output result, due to the complexities of designing both the database itself and the query system for reading items from the database. Spectral database 112 incorporates a number of features in order to enable faster reading of the spectral database and identification of the spectra. Spectral database 112 may also include unknown spectra associated with previous scans or queries.

Spectral database 112 may be a document-based database. Each document in spectral database 112 is stored in a substantially flat-file structure, such that there is zero or minimal change to the underlying data in storage. Spectral database 112 may be accessed by multiple components or services at once and in turn provide a continuous stream of compressed files in response to multiple simultaneous queries. Access to spectral database 112 may be accomplished directly by each of the query services 110 or may be through an interface, such as a file reading API. Spectral database 112 may be configured such that use of known file-reading APIs is supported without change to the underlying code.

Spectral database 112 may provide the ability to select files in any manner as appropriate for the data received from the mass spectrometer 102. For example, often a sample is run through a chromatographic system prior to being run through the mass spectrometer which introduces a time-based element into the data and finding a match for this data may involve selecting comparison files from the spectral database 112 in a time-based manner. Data may also be received from the mass spectrometer which does not include such a time-based element, and in such cases it may be desirable to select files from the spectral database in an experiment-based or some other manner.

Spectral database 112 may also be configured for random-read access, such as by being configured as a document-based database. Random-read access provides for faster reading and response than a more traditional sequential read system. Spectral database 112 may have access to all scans or files in the database instantaneously and at all times. Spectral database 112 may be a distributed database, using, for example, Cassandra; a document database, using, for example, MongoDB; or proprietary implementations of such databases in cloud platforms such as AWS, Google, or Azure. In examples, a query service 110 that submits a query to the spectral database 112 may then itself have instant access to all files or scan in the database.

Results database 114 receives and collects the matches from each of the query service 110. In examples, results database 114 may communicate with readout database 104.

In some envisioned system configurations, results database 114 may determine the overall speed of the system 100. In systems relying on sequential writing of results, write speed of the matches determined and the output format creates a bottleneck in the workflow that delays the production of the final system output. By introducing a database, or other storage component, into the system to receive and hold the matches from the query services 110 and provide those matches to a results service 116 which formats the matches into a complete output format.

Results service 116 retrieves the matches from the results 114 and formats or transforms the matches into an output data structure. Results service 116 may generally apply known forms of extract-transform-load (ETL) formatting to the matches to produce the output data structure. In examples, results service 116 receives or retrieves the matches directly from the query services 110.

User interface 118 receives the output data structure from results service 116 and permits the output data structure to be displayed to a user. User interface 118 may be any known or contemplated display device, including but not limited to monitors, laptops, tablets, mobile phones, etc. User interface 118 may be associated or in communication with mass spectrometer 102, or user interface 118 may be a fully independent component or system. User interface 118 and mass spectrometer 102 may occupy a common physical space or may be physically distant from one another. User interface 118 may represent a fully virtual machine. User interface 118 may generally be separate from or outside of system 100, and receive data, such as the output data structure, from the system 100 through an API gateway or another reverse proxy that provides traffic routing and management of access policies for the system 100.

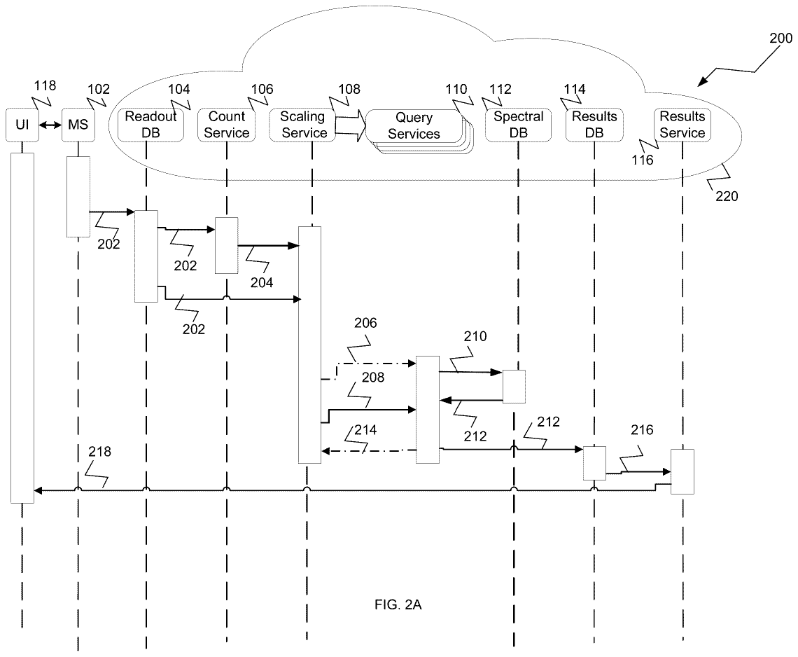

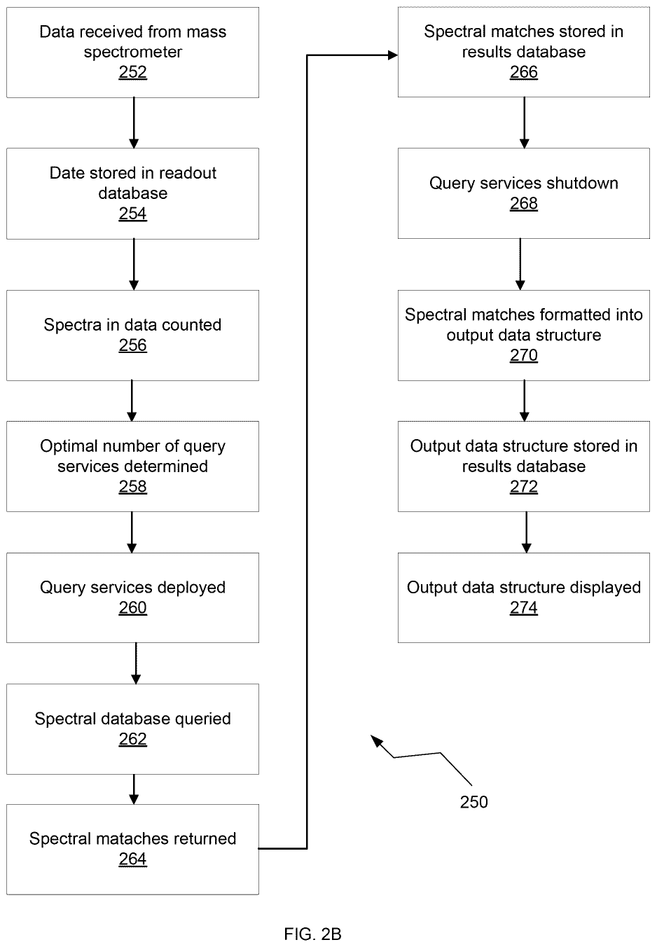

FIG. 2 depicts dataflows and system procedures 200 for analyzing a spectral scan from a mass spectrometer, such as one or more mass spectrometer 102 discussed in reference to FIG. 1, above. Referring back to FIG. 1, the components used to implement procedure 200 include the readout database 104, the count service 106, the scaling service 108, the plurality of query services 110, the spectral database 112, the results database 114, and the results service 116. As discussed above, not all of the components are required to practice the invention, and variations in the arrangement and type of the components may be made without departing from the spirit or scope of the invention. For example, the system may further incorporate an initiation or workflow management service to further distribute the services and increase the flexibility of the system or the functions within the system may be redistributed among the services, such as by configuring the scaling service 108 to execute the count function and thereby excluding the counter service 106.

Components of the system may be fully virtual and physically distributed, or components may have differing configurations. For example, mass spectrometer 102 and user interface 118 may represent physical machines, which may occupy a common physical space or physically distant spaces. Other components and service of the system, such databases 104, 112, 114 and services 106, 108, 110, 116 may be fully virtual and “occupy” a cloud space 220, with underlying physical hardware occupying a common physical space with mass spectrometer 102 or user interface 118, or distant from both, or distributed across one or more physical spaces.

In an example, scan output data 202 is produced by a mass spectrometer 102 and received by readout database 104. Count service 106 retrieves scan output data 202 from readout database 104 and determines a number of spectra 204 within the scan output data 202. Scaling service 108 retrieves the scan output data 202 from the readout database 104. Scaling service 108 also receives or retrieves the number of spectra 204 from the count service 106. In examples, count service 106 may send the number of spectra 204 to the readout database 104 to be stored, and scaling service 108 may instead retrieve the number of spectra 204 from the readout database 104.

Scaling service 108 divides the scan output 202 into divided spectra 208 according to the number of spectra 204. Scaling service 108 initiates 206 a number of query services 110 according to the number of spectra 204. Scaling service 108 may distribute the divided spectra 208 among the query services 110. In examples, query services 110 may instead each retrieve a divided spectra 208 from the scaling service 108 or from the readout database 104. Initiation 206 of query services 110 may include generation and assignment of specific protocols for each query service 110 identifying one or more of divided spectra 208 for which the particular query service 110 should seek a match.

Query services 110 each query spectral database 112 with the particular spectra 210 of the divided spectra 208 to obtain a match 212 for the spectra 210. As each query service 110 obtains a match 212 for the submitted spectra 210, query services 110 may evaluate whether match 212 meets a match threshold of similarity to the submitted spectra 210.

Query services 110 may each access the spectral database 112 simultaneously. In examples, query services 110 may include protocols dictating a sequential or staggered order for accessing spectral database 112. For example, some scan output data 202 may not be evenly divisible and divided spectra 208 may have spectra of various sizes to be identified. Scaling service 108 may provide protocols to query services 110 so that a particular query service 110 with a larger spectra will initiate query and identification before another query service 110 with a smaller spectra.

Match 212 may comprise any appropriate determination of match between the query spectra 210 and a known spectra from the spectral library 112. Match 212 may include a calculation, which may be performed by query services 110, for a match value. The match value may, for example, be a fit value or a purity value. Match 212 is stored in results database 114 by query services 110. Once match 212 is stored, query services 110 are scaled back and return 214 to scaling service 108. Query services 110 may execute returning 214 to scaling service 108 by reintegrating themselves with scaling service 108. Scaling service 108 may execute the return 214 of query services 110 by reclaiming query services 110, such as by terminating a map state.

In examples, results database 114 may communicate with readout database 104 to confirm common metadata between scan output 202 and matches 212, each of which is associated with at least one of divided spectra 208. In examples, results service 116 may instead perform the confirmation.

Results service 116 retrieves stored matches 212 from results database 114 and performs output processing 216. Output processing 216 is generally extract-transform-load (ETL) processing. Those skilled in the art will generally be familiar with one or more forms of ETL processing which may be appropriate for output processing 216. Results service 116 may also utilize results database 114 for storage of intermediate processing stages of output processing 216. In examples, results service 116 may also communicate with readout database 104 to confirm metadata identities by scan output 202 and matches 212, each of which is associated with at least one of divided spectra 208.

When output processing 214 is complete, results service 116 may store the final output product 218 in results database 114 or may direct final output product 218 directly to a user interface 118. In examples, final output product 218 may be stored in another database, such as a database integrated with or dedicated to user interface 118 or mass spectrometer 102.

Referring now to FIG. 2B, an example process 250 of a data analysis as executed by a system according to the present disclosure, such as system 100 of FIG. 1. Example process 250 may be implemented using example dataflow and processes 200 of FIG. 2A.

Data is received from the mass spectrometer 252 and stored in a readout database 254. Data may be received directly from mass spectrometer 252 or via a network. In embodiments, data from mass spectrometer 252 may be directed to the system by a gateway or other application programming interface. Data from mass spectrometer 252 may be stored directly in readout database 254 or may be received and processed directly by system modules. Data from mass spectrometer may be sent directly to readout database 254 by mass spectrometer 252 or may be directed to readout database 254 by an intermediate module, such as an application programming interface.

The number of spectra in the data is counted 256 and an optimal number of query services is determined 258 according to the number of spectra in the data. One or more query services are deployed 260, according to the optimal number of query services determined. The optimal number of query services may be determined such that each query service receives a single spectra from the data. A single spectra may comprise a single peak. A single spectra may comprises two or more associated peaks. A single spectra may comprise a known or possible pattern of peaks. The optimal number of query services may be determined such that some of the query services receive a single spectra and some of the query services receive one or more spectra. For example, a query service may receive one or more simple or well-known peaks or spectra. A query service may receive a group of spectra with features indicating association among the group. A query service may receive two or more spectra with features indicating overlap or common source.

Each of the one or more query services deployed receives one or more of the number of spectra. The spectral database is queried 262 to determine a match for each of the one or more of the number of spectra. One or more spectral matches are returned 264 for each of the one or more of the number of spectra. Each query service may return a single match for a spectra or one or more matches if a single high-confidence match is not found. A query service may return a range or selection of possible matches. Query services may return a preferred match with alternative match possibilities. Each spectral match is stored in a results database 266 and each query service is shutdown 268 once the spectral match is stored.

Each of the spectral matches is formatted into an output data structure 270. Each of the spectral matches may be combined such that a single output data structure encompasses all the spectral matches returned for each of the number of spectra in the original data output by the mass spectrometer. The output data structure may be a single output, or may be two or more possible output data structures. For example, if a spectra has two or more possible matches, two or more output data structures may be produced according to the alternative possible matches. The output data structure is stored in the results database 272 and may be displayed 274, such as on a user interface.

Aspects of the present disclosure may operation as a container-based system which may be implemented on top of a container management layer containing a container management system. In examples, the container management system comprises a Kubernetes system, though other management systems are contemplated and may be applied to the subject matter of the present disclosure.

Other container management systems with functionality similar to Kubernetes may also be used, and specific reference to Kubernetes is by example only. In embodiments, the container orchestration engine may be a Kubernetes container runtime.

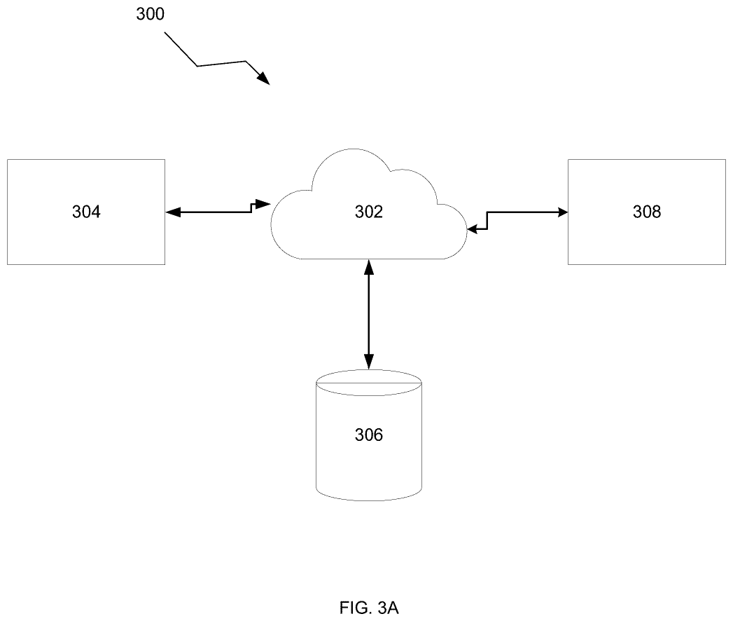

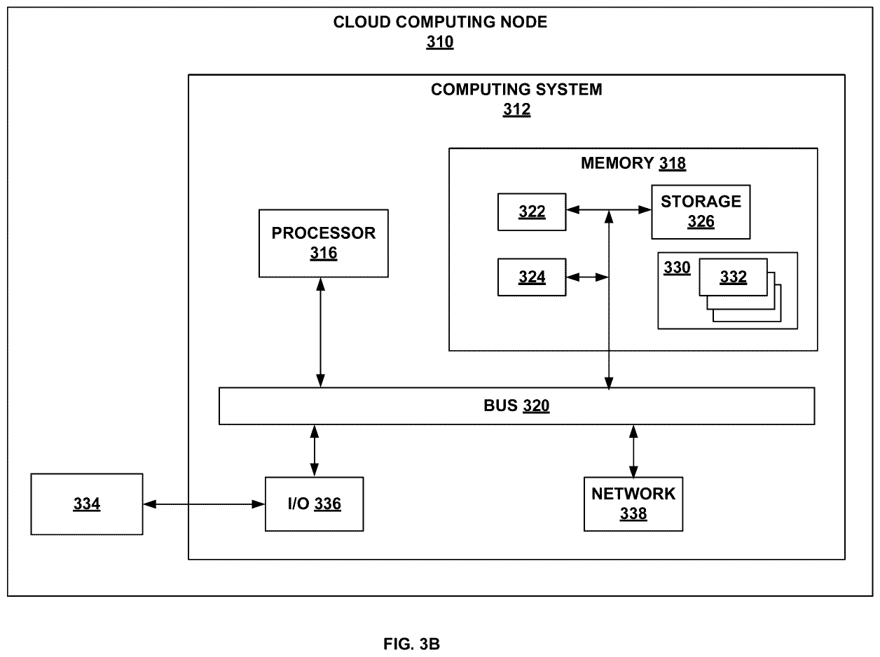

Referring now to FIG. 3A, a diagram of an example data processing environment 300 is provided in which the illustrative examples of the present disclosure may be implemented. FIG. 3 is only meant as an example and is not intended to assert or imply any limitation to the environments in which different examples of the present disclosure may be implemented. Many modifications to the depicted environments may be made. Example system 100, example dataflow 200, and example process 250 may be implemented in a data processing environment such as example data processing environment 300.

FIG. 3A is a network system 300 which may include a network of computers, data processing systems, and other devices in which the illustrative examples may be implemented. Network system 300 contains network 302, which may be used to provide communications links between computers, data processing systems, and other devices connected together within network system 300. Network 302 may include connections, such as, for example, various wired or wireless communication links.

Server 304 connects to network 302, along with storage 306. Server 304 may provide a set of services corresponding to a microservice architecture comprising a plurality of different microservices. Server 304 may represent a plurality of servers hosting different microservice architectures that perform different services. Server 304 may be a set of one or more cloud computing nodes with which local computing devices used by cloud consumers, such as, for example, client 308, may communicate. The cloud computing nodes may communicate with one another and may be grouped physically or virtually into one or more networks, such as private, community, public, or hybrid clouds as described hereinabove, or a combination thereof. This allows system 300 to offer infrastructure, platforms, or software as services for which a cloud consumer does not need to maintain resources on a local computing device.

Client 308 also connects to network 302. Client 308 is a client or clients of server 304. Client 308 may represent a plurality of workstations corresponding to a plurality of different users. The users may be, for example, application developers or users of microservice architectures. Client 308 may also be a cloud computing node.

Server 304 may provide information, such as software applications and programs to client 308. Client 308 may represent a local computing environment, such as a desktop computer, a laptop computer, handheld computer, and the like, that may run a locally deployed microservice of a microservice architecture. Respective users of client 308 may deploy a microservice in a software development kit operating on client 308 for development of one or more functions of a locally deployed microservice.

Storage 306 is a network storage device capable of storing any type of data in a structured format or an unstructured format. Storage 306 may represent a plurality of network storage devices. Storage 306 may store identifiers and uniform resource locators for a plurality of client devices, identifiers and uniform resource locators for a plurality of servers in a remote-computing environment, a plurality of different microservice architectures, microservice source code, software development kits, and the like. Storage 306 may store other types of data, such as authentication or credential data that may include user names, passwords, and biometric data associated with application developers and system administrators, for example.

Network system 300 may include any number of additional servers, clients, storage devices, and other devices not shown. Program code located in network data processing system 300 may be stored on a computer readable storage medium and downloaded to a computer or other data processing device for use. For example, program code may be stored on a computer readable storage medium on server 304 and downloaded to client 308 over network 302 for use on client 308.

In the depicted example, network system 300 may be implemented as a number of different types of communication networks, such as, for example, an internet, an intranet, a local area network (LAN), and a wide area network (WAN). FIG. 3A is intended as an example only, and not as an architectural limitation for the different illustrative examples.

Referring now to FIG. 3B, a block diagram of an example of a cloud computing node is shown, upon which aspects of the present disclosure may be implemented. Cloud computing node 310 is only one example of a suitable cloud computing node and is not intended to suggest any limitation as to the scope of use or functionality of embodiments of the invention described herein. Regardless, cloud computing node 310 is capable of being implemented and performing any of the functionality set forth hereinabove.

In cloud computing node 310, there is computer system 312, which works with other general purpose or special purpose computing system environments or configurations. Examples of well-known computing systems, environments, or configurations that may be suitable for use with computer system 312 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices.

Computer system 312 may be described in the general context of computer system-processing instructions, such as program modules, being processed by a computer system. Generally, program modules may include routines, programs, objects, components, logic, data structures, and so on that perform particular tasks or implement particular abstract data types. Computer system 312 may be practiced in distributed cloud computing environments where tasks are performed by remote processing devices that are linked through a communications network. In a distributed cloud computing environment, program modules may be located in both local and/or remote computer system storage media including memory storage devices.

As depicted in FIG. 3B, computer system 312 in cloud computing node 310 is shown in the form of a general-purpose computing device. The components of computer system 312 may include, but are not limited to, one or more processors 316, memory 318, and bus 320 that couples various system components, including memory 318, to processor 316.

Processor 316 processes instructions for software that may be loaded into memory 318. Processor 316 may be a number of processors, a multi-processor core, or some other type of processor, depending on the particular implementation. Further, processor 316 may be implemented using one or more different processor systems in which a main processor is present with secondary processors, and my be on a single chip. In another example, processor 316 may be a symmetric multi-processor system containing multiple processors of the same type.

Bus 320 represents one or more of any of several types of bus structures, including a memory bus or memory controller, a peripheral bus, an accelerated graphics port, and a processor or local bus using any of a variety of bus architectures. By way of example and not limitation, such architectures include Industry Standard Architecture (ISA) bus, Micro Channel Architecture (MCA) bus, Enhanced ISA (EISA) bus, and Peripheral Component Interconnects (PCI) bus.

Computer system 312 may include a variety of computer system readable media. Such media may be any available media that is accessible by computer system 312 and includes both volatile and non-volatile media and removable and non-removable media.

Memory 318 can include computer system readable media in the form of volatile memory, such as random access memory (RAM) 322 and/or cache 324. Computer system 312 may further include other removable/non-removable, volatile/non-volatile computer system storage media. By way of example only, storage system 326 can be provided for reading from and writing to a non-removable, non-volatile magnetic media, such as a hard drive. Although not shown, a magnetic disk drive for reading from and writing to a removable, non-volatile magnetic disk, and an optical disk drive for reading from or writing to a removable, non-volatile optical disk, or other optical media can be provided. In such instances, each can be connected to bus 320 by one or more data media interfaces. Memory 318 may include at least one program product having a set of program modules that are configured to carry out the functions of embodiments of the invention. As used herein, a set, when referring to items, means one or more items. For example, a set of program modules is one or more program modules.

Program 330, having a set of program modules 332, may be stored in memory 318, by way of example, as well as an operating system, one or more application programs, other program modules, and program data. Each of the operating systems, one or more application programs, other program modules, and program data or some combination thereof, may include an implementation of a networking environment. Program modules 332 generally carry out the functions and/or methodologies of embodiments of the invention as described herein.

Computer system 312 may also communicate with one or more external devices 334, such as a keyboard, a mouse, a display, or one or more other devices to enable a user to interact with computer system 312. External devices 334 may further include any devices (e.g., network card, modem, etc.) that enable computer system 312 to communicate with one or more other computing devices. These communication can occur via I/O interface 336. Computer system 312 can communicate with one or more networks, such as a local area network (LAN), a general wide area network (WAN), or a public network, such as the Internet via network adapter 338.

Network adapter 338 communicates with other components of computer system 312 via bus 320. Other hardware and/or software components, which may not be depicted in FIG. 3B, are able to be used with computer system 312. Examples include, but are not limited to, microcode, device drivers, redundant processor units, external disk drive arrays, RAID systems, tape drives, and data archival storage systems, etc.

It should be understood that the figures and examples presented herein are for example purposes only. The architecture of the example examples presented herein is sufficiently flexible and configurable, such that it may be utilized (and navigated) in ways other than that shown in the accompanying figures.

The above specification, examples and data provide a complete description of the manufacture and use of the composition of the invention. Since many examples of the invention can be made without departing from the spirit and scope of the invention, the invention resides in the claims hereinafter appended.

1. A analysis system for data from a mass spectrometer, comprising:
a spectral database;
a count service for:
receiving scan data generated by the mass spectrometer, and identifying a number of spectra in the data;
a scaling service for:
receiving the scan data generated by the mass spectrometer,
receiving the number of spectra from the count service, and
initiating a plurality of query services, each query service of the plurality of query services corresponding to at least one spectra of the number of spectra, each query service of the plurality of query services for:
querying the spectral database with the corresponding at least one spectra, and
returning a match between the corresponding at least one spectra and at least one known spectra from the spectral database; and
a results service for:
retrieving each match, and
formatting each match into an output data structure.

2. The data analysis system of claim 1, further comprising a scan output database for receiving the scan data generated by the mass spectrometer.

3. The analysis system of claim 2, wherein the scan data generated by the mass spectrometer comprises a plurality of key-value pairs.

4. The analysis system of claim 3, wherein each of the plurality of key-value pairs comprises a scan and an offset.

5. The analysis system of claim 1, wherein the spectral database comprises a document-based database.

6. The analysis system of claim 1, wherein the count service performs signal processing on the scan data generated by the mass spectrometer.

7. The analysis system of claim 6, wherein the count service is further for executing a peak extraction on the data collected by the mass spectrometer such that the number of spectra coincides with a number of peaks in the data.

8. The data analysis system of claim 1, wherein the at least one spectra of the number of spectra is only one spectra of the number of spectra.

9. The data analysis system of claim 1, wherein the match comprises a fit value.

10. The data analysis system of claim 1, wherein the match comprises a purity value.

11. The data analysis system of claim 1, further comprising a results database,
wherein each query service stores the match corresponding to its at least one spectra in the results database.

12. The data analysis system of claim 11, wherein the results service stores the output data structure in the results database.

13. The data analysis system of claim 11, further comprising a scan output database for receiving the scan data generated by the mass spectrometer,
wherein the scan data comprises a plurality of paired sets of a scan and an offset; and
wherein the results database communicates with the scan output database to associate each match with one paired set of the plurality of paired sets of a scan and an offset.

14. The data analysis system of claim 11, wherein each query service, in response to storing the match associated with its at least one spectra in the results database, is reclaimed by the scaling service.

15. The data analysis system of claim 1, wherein formatting each match into an output data structure comprises extract-transform-load processing.

16. The data analysis system of claim 1, wherein the mass spectrometer comprises a tandem mass spectrometry system.

17. A method of analysis of data collected by a mass spectrometer, the method comprising:
receiving data collected by the mass spectrometer;
counting a number of spectra in the data;
initiating a plurality of query services, each query service of the plurality of query services corresponding to at least one spectra of the number of spectra;
querying, by each of the plurality of query services, a spectral database;
returning, by each of the plurality of query services, at least one match between the
corresponding at least one spectra and a known spectra from the spectral database; formatting each match from each of the plurality of query services into an output data structure; and
storing the output data structure in a results database.

18. The method of analysis of claim 17, wherein the mass spectrometer comprises a tandem mass spectrometry system.

19. The method of analysis of claim 17 further comprising:
storing, by each of the query services, the match associated with its at least one spectra in the results database; and
reclaiming, by the scaling service, each of the plurality of query services.

20. The method of analysis of claim 17, where each match from each of the plurality of query services comprises at least one of a fit value and a purity value.
